(19) United States
(12) Patent Application Publication
(10) Pub. No.: US 2012/0115158 A1
(43) Pub. Date: May 10, 2012

(54) METHOD TO DETERMINE EMBRYO AND OOCYTE QUALITY BASED ON CERAMIDASE

(75) Inventors: Edward H. Schuchman, Haworth, NJ (US); Efrat Eliyahu, New York, NY (US)

(73) Assignee: MOUNT SINAI SCHOOL OF MEDICINE, New York, NY (US)

(21) Appl. No.: 13/318,377

(22) PCT Filed: May 3, 2010
(86) PCT No.: PCT/US2010/033422

§ 371 Date: Jan. 23, 2012

Related U.S. Application Data
(60) Provisional application No. 61/174,823, filed on May 1, 2009.

(51) Int. Cl. C12Q 1/34; C12Q 1/68; G01N 33/573

(52) U.S. Cl. 435/6.18; 435/18; 435/7.1

(57) ABSTRACT

The present invention relates to a method of predicting the likelihood of embryo or oocyte survival. This method involves providing a sample of an embryo, an oocyte, or surrounding liquid or cells, and screening the sample for ceramidase expression or activity. The ceramidase expression or activity obtained through said screening is then correlated to a prediction of the likelihood of embryo or oocyte survival. Also disclosed is a kit.

This application claims benefit of U.S. Provisional Patent Application Ser. No. 61/174,823, filed May 1, 2009, which is hereby incorporated by reference in its entirety.

This invention was made with government support under grant number R01 DK54830 awarded by The National Institutes of Health. The government has certain rights in this invention.

### FIELD OF THE INVENTION

The present invention relates to a method of determining embryo and oocyte quality based on ceramidase expression or activity.

### BACKGROUND OF THE INVENTION

Due to its involvement in the human genetic disorder Farber Lipogranulomatosis (“FD”), acid ceramidase (“AC;” N-acylsphingosine deacylase, I.U.B.M.B. Enzyme No. EC 3.5.1.23) is the most extensively studied member of the ceramidase enzyme family. The protein has been purified from several sources, and the human and mouse cDNAs and genes have been obtained (Bernardo et al., “Purification, Characterization, and Biosynthesis of Human Acid Ceramidase,” J. Biol. Chem. 270:11098-102 (1995); Koch et al., “Molecular Cloning and Characterization of a Full-length Complementary DNA Encoding Human Acid Ceramidase. Identification of the First Molecular Lesion Causing Farber Disease,” J. Biol. Chem. 2711:33110-5 (1996); Li et al., “Cloning and Characterization of the Full-length cDNA and Genomic Sequences Encoding Murine Acid Ceramidase,” Genomics 50:267-74 (1998); Li et al., “The Human Acid Ceramidase Gene (ASAH): Chromosomal Location, Mutation Analysis, and Expression,” Genomics 62:223-31 (1999)). Growing interest in the biology of this and other ceramidases stems from the fact that these enzymes play a central role in ceramide metabolism. Ceramide is a signaling lipid that is produced in response to various stimuli (Hannun, “Function of Ceramide in Coordinating Cellular Responses to Stress,” Science 274:1855-9 (1996); Spiegel et al., “Signal Transduction Through Lipid Second Messengers,” Curr. Opin. Cell. Biol. 8:159-67 (1996)). Normally present in low amounts, in response to these factors, ceramide is rapidly produced at the cell surface, leading to membrane re-organization and downstream signaling that results in apoptosis. After stimulation, AC and/or other ceramidases may then hydrolyze ceramide into the individual fatty acid and sphingosine components (Gatt, “Enzymic Hydrolysis and Synthesis of Ceramide,” J. Biol. Chem. 238:3131-3 (1963); Gatt, “Enzymatic Hydrolysis of Sphingolipids. 1. Hydrolysis and Synthesis of Ceramides by an Enzyme from Rat Brain,” J. Biol. Chem. 241:3724-31 (1966); Hassler & Bell, “Ceramidase: Enzymology and Metabolic Roles,” Adv. Lip. Res. 26:49-57 (1993)). Because ceramide degradation is the only source of intracellular sphingosine (Rother et al., “Biosynthesis of Sphingolipids: Dihydroceramide and Not Sphinganine Is Desaturated by Cultured Cells,” Biochem. Biophys. Res. Commun. 189:14-20 (1992)), these enzymes may also be rate-limiting steps in determining the intracellular levels of this compound. Importantly, a derivative of sphingosine, sphingosine-1-phosphate (“S1P”), can counteract the apoptotic effects of ceramide (Cuvillier et al., “Suppression of Ceramide-mediated Programmed Cell Death by Sphingosine-1-phosphate,” Nature 381:800-3 (1996)), leading to the suggestion that ceramidases can be “rheostats” that maintain a proper balance between cell growth and death (Spiegel & Merrill, “Sphingolipids Metabolism and Cell Growth Regulation,” FASEB J. 10:1388-97 (1996)).

Ovulated eggs undergo molecular changes characteristic of apoptosis unless successful fertilization occurs (Marston & Chang, “The Fertilizable Life of Ova and Their Morphology Following Delayed Insemination in Mature and Immature Mice,” J. Exp. Zool. 155:237-52 (1964); Tarin et al., “Long-term Effects of Postovulatory Aging of Mouse Eggs on Offspring: A Two-generational Study,” Biol. Reprod. 61:1347-55 (1999)). While multiple factors, including ceramide, have been characterized as pro-apoptotic elements involved in this process (Perez et al., “A Central Role for Ceramide in the Age-related Acceleration of Apoptosis in the Female Germline,” FASEB J. 19:860-2 (2005); Miao et al., “Cumulus Cells Accelerate Aging of Mouse Oocytes,” Biol. Reprod. 73:1025-1031 (2005); Kerr et al., “Morphological Criteria for Identifying Apoptosis,” in 1 CELL BIOLOGY: A LABORATORY HANDBOOK 319-29 (Julio E. Celis ed., 1994); Gordo et al., “Intracellular Calcium Oscillations Signal Apoptosis Rather Than Activation in in Vitro Aged Mouse Eggs,” Bio. Reprod. 66:1828-37 (2002)), little is known about factors that sustain egg or embryo survival.

The present invention is directed to overcoming these and other deficiencies in the art.

### SUMMARY OF THE INVENTION

The present invention relates to a method of predicting the likelihood of embryo or oocyte survival. This method involves providing a sample of an embryo, an oocyte, or surrounding liquid or cells, and screening the sample for ceramidase expression or activity. The ceramidase expression or activity obtained through said screening is then correlated to a prediction of the likelihood of embryo or oocyte survival.

The present invention also relates to a kit for predicting the likelihood of embryo or oocyte survival. The kit includes an agent which specifically recognizes ceramidase expression or activity and a label which detects said recognition of ceramidase expression or activity by said agent.

The present invention demonstrates that ceramidase is a normal component of developing embryos and oocytes that can be measured in culture media. Furthermore, ceramidase expression in embryo and oocyte culture media can be used to predict their quality.

The present invention also demonstrates that AC is one factor required for early embryo and oocyte survival. Gene targeting has been used to inactivate the AC gene (Asah1) in mice (Li et al., “Insertional Mutagenesis of the Mouse Acid Ceramidase Gene Leads to Early Embryonic Lethality in Homozygotes and Progressive Lipid Storage Disease in Heterozygotes,” Genomics 79:218-24 (2002), which is hereby incorporated by reference in its entirety). Initial characterization of these animals revealed that heterozygous mice (“Asah1+/−”) had a progressive lipid storage disease phenotype, and that a complete loss of AC activity led to the absence of mutant individuals. It remained unclear, however, whether the Asah1−/− embryos were not formed, or, alternatively, if they were formed, whether they died during early embryogenesis.

The present invention also describes the use of a combination of molecular, biochemical, and morphological methods to follow the development of individual embryos obtained from Asah1+/− intercrosses. These analyses showed that Asah1−/− embryos could be formed, but underwent apoptotic death at the 2-cell stage. It was also demonstrated that Asah1 is one of the earliest genes expressed in newly formed embryos. Further, AC is shown to be a predominant protein in unfertilized eggs, and expression of this protein and gene is decreased during egg aging unless fertilization occurs. Overall, these results demonstrate that AC is an essential component of newly formed embryos, and is required for their survival beyond the 2-cell stage.

Acid ceramidase has a natural ability to enter cells through mannose receptors and/or mannose-6-phosphate receptors located on various cell types, including oocytes, neurons, and synovial fibroblasts. Additionally, cells that do not have these receptors contain “scavenging” receptors that can lead to internalization of AC. This implies that administering acid ceramidase into a culture medium can increase the level of the enzyme inside cells in the culture, leading to a reduction in ceramide levels within the cell. It also appears that acid ceramidase can cross the zona pellucida of oocytes, something most molecules cannot do.

Increasing ceramide levels in cells almost always leads to cell death, and ceramidases are the only enzymes that can hydrolyze ceramide. Expression of acid ceramidase in cells has at least two consequences: removal of ceramide, and the production of sphingosine and sphingosine-1-phosphate (two well-characterized, anti-apoptotic lipids). Therefore, without being bound by theory, it is expected that acid ceramidase promotes cell survival in at least two ways: by removing ceramide and by producing sphingosine and sphingosine-1-phosphate. Acid ceramidase is the only known molecule that does both of these. A measurement of the activity level of this enzyme in the embryonic cell or an egg or surrounding fluids or cells (e.g. cumulus cells), can be important in the prediction of the embryo and oocyte quality.

### BRIEF DESCRIPTION OF THE DRAWINGS

FIG. 1 is a graph of AC activity and a representative western blot (inset) of cell extracts from unfertilized mouse eggs. Cell extracts from 400 pooled eggs were analyzed by western blot (see Example 10). A goat anti-human AC IgG was used to detect the murine AC precursor protein (55 kDa) and AC β-subunit (40 kDa). For AC activity assays, cell extracts were prepared from 65 pooled eggs, incubated for 22 hours at 37° C. with boron-dipyrromethene (“BODIPY”)-conjugated C12-ceramide, and then analyzed by HPLC. The AC activity in these extracts was significantly higher in comparison to the blank control (t-test, p<0.005). These data show that AC is expressed at high levels in unfertilized, healthy mouse eggs. The data represent mean±S.E.M; n=3 independent experiments.

FIGS. 2A-G are representative immunohistochemistry images of fixed, unfertilized eggs. Goat IgG was used against human AC (FIGS. 2A and 2D), and rat IgG was used against the lysosomal marker Lamp1 (FIGS. 2B and 2E). FIGS. 2C and 2F show merged images. Localization of the primary antibodies was visualized using a fluorescent secondary antibody (Cy-3/2) and laser-scanning confocal microscopy (see Example 5). Eggs labeled only with secondary antibodies were used as a control (FIG. 2G). Bar=10 μm. The data represent three independent experiments, and confirm that AC is expressed at high levels in healthy mouse eggs.

FIGS. 3A-P are stained images of denuded and fixed human oocytes at the germinal vesicle stage (“GV”), germinal vesicle breakdown stage (“GVBD”), MI stage, or MII stage. The oocytes were incubated with polyclonal anti-AC antibody (FIGS. 3C, 3G, 3K, and 3O), polyclonal anti-LAMP antibody (FIGS. 3B, 3F, 3J, and 3N), or the Hoechst DNA-specific fluorochrome 33342 (FIGS. 3A, 3E, 3I, and 3M). FIGS. 3D, 3H, 3L, and 3P show the preceding three images superimposed upon each other (“Merge”), to identify the co-localization of AC with LAMP and/or the cellular DNA. Bar=10 μm. These data are the first to demonstrate expression of AC in human oocytes.

FIGS. 4A-D are stained images of denuded and fixed low grade human embryos. The embryos were incubated with the Hoechst DNA-specific fluorochrome 33342 (FIG. 4A), anti-acid sphingomyelinase antibody (“ASM”) (FIG. 4B), or polyclonal anti-AC antibody (FIG. 4C). FIG. 4D shows FIGS. 4A-C superimposed upon each other (“Merge”), to identify the co-localization of AC with DNA and/or ASM. Localization of the primary antibodies was imaged using secondary antibodies Cy-3 or Cy-2 and Laser-scanning confocal microscopy. Embryos were graded according to the morphology of the inner and outer cell masses. The data represent three independent experiments. These data are the first to demonstrate expression of AC in human embryos.

FIGS. 5A-D are stained images of denuded and fixed high grade human embryos. The embryos were incubated with the Hoechst DNA-specific fluorochrome 33342 (FIG. 5A), anti-ASM antibody (FIG. 5B), or polyclonal anti-AC antibody (FIG. 5C). FIG. 5D shows FIGS. 5A-C superimposed upon each other (“Merge”), to identify the co-localization of AC with DNA and/or ASM. Localization of the primary antibodies was imaged using secondary antibodies Cy-3 or Cy-2 and Laser-scanning confocal microscopy. Embryos were graded according to the morphology of the inner and outer cell masses. The data represent three independent experiments.

FIG. 6 is a western blot of human follicular fluid samples using antibodies against the AC precursor protein or the AC α-subunit. A 1 μl sample was loaded onto lane 1 (“FF 1λ”) and a 10 μl sample was loaded onto lane 2 (“FF 10λ”). Pure AC was loaded onto lane 3 as a control (”Con AC″).

FIG. 7 is a plot of AC activity in human follicular fluid as a function of maternal age (in years).

### DETAILED DESCRIPTION OF THE INVENTION

The present invention relates to a method of predicting the likelihood of embryo or oocyte survival. This method involves providing a sample of an embryo, an oocyte, or surrounding liquid or cells and screening the sample for ceramidase expression or activity. The ceramidase expression or activity obtained through said screening is then correlated to a prediction of the likelihood of embryo or oocyte survival.

Ceramidases hydrolyze the amide linkage of ceramides to generate free fatty acids and sphingoid bases (Nikolova-Karakashian et al., Methods Enzymol. 311:194-201 (2000), Hassler et al., Adv. Lipid Res. 26:49-57 (1993), which are hereby incorporated by reference in their entirety). There are three types of ceramidases described to date (Nikolova-Karakashian et al., Methods Enzymol. 311:194-201 (2000), which is hereby incorporated by reference in its entirety). These are classified as acid, neutral, and alkaline ceramidases according to their pH optimum of enzymatic activity.

Acid ceramidases have optimal enzymatic activity at a pH of 1-5. The murine acid ceramidase was the first ceramidase to be cloned (Koch et al., J. Biol. Chem. 271:33110-33115 (1996), which is hereby incorporated by reference in its entirety). It is localized in the lysosome and is mainly responsible for the catabolism of ceramide. Dysfunction of this enzyme because of a genetic defect leads to a sphingolipidosis disease called Farber disease (Koch et al., J. Biol. Chem. 271:33110-33115 (1996), which is hereby incorporated by reference in its entirety).

The neutral ceramidases have been purified from rat brain (El Bawab et al., J. Biol. Chem 274:27948-27955 (1999), which is hereby incorporated by reference in its entirety) and mouse liver (Tani et al., J. Biol. Chem 275:3462-3468 (2000), which is hereby incorporated by reference in its entirety), and recently they were cloned from Pseudomonas (Okino et al., J. Biol. Chem. 274:36616-36622 (1999), which is hereby incorporated by reference in its entirety), mycobacterium (Okino et al., J. Biol. Chem. 274:36616-36622 (1999), which is hereby incorporated by reference in its entirety), mouse (Tani et al., J. Biol. Chem 275:11229-11234 (2000), which is hereby incorporated by reference in its entirety), and human (El Bawab et al., J. Biol. Chem. 275:21508-21513 (2000), which is hereby incorporated by reference in its entirety). These ceramidases share significant homology, and this homology extends to putative proteins deduced from expressed sequence tag (EST) sequences of Dictyostelium discoideum and Arabidopsis thaliana (Okino et al., J. Biol. Chem. 274:36616-36622 (1999), El Bawab et al., J. Biol. Chem. 275:21508-21513 (2000), which are hereby incorporated by reference in their entirety). These ceramidases have a broad pH optimum ranging from 5 to 9 for their activity (Tani et al., J. Biol. Chem 275:11229-11234 (2000), El Bawab et al., J. Biol. Chem. 275:21508-21513 (2000), which are hereby incorporated by reference in their entirety). They appear to hydrolyze unsaturated ceramide preferentially, saturated ceramide (dihydroceramide) slightly, and hardly hydrolyze phytoceramide (Tani et al., J. Biol. Chem 275:11229-11234 (2000), which is hereby incorporated by reference in its entirety). The Pseudomonas, mouse, and human neutral ceramidases have a reverse ceramidase activity of catalyzing the formation of ceramide from sphingosine and a fatty acid (Okino et al., J. Biol. Chem. 274:36616-36622 (1999), Tani et al., J. Biol. Chem 275:11229-11234 (2000), Kita et al., Biochim. Biophys. Acta 1485:111-120 (2000), which are hereby incorporated by reference in their entirety). El Bawab et al. (El Bawab et al., J. Biol. Chem. 275:21508-21513 (2000), which is hereby incorporated by reference in its entirety) have shown previously that the human neutral ceramidase is localized in the mitochondria.

Alkaline ceramidases have optimal activity at a pH of 9-14. Two alkaline ceramidases were purified from Guinea pig skin epidermis. These two enzymes were membrane bound, and their estimated molecular masses on SDS-PAGE were 60 and 148 kDa, respectively (Yada et al., “Purification and Biochemical Characterization of Membrane-Bound Epidermal Ceramidases from Guinea Pig Skin,” J. Biol. Chem. 270:12677-12684 (1995), which is hereby incorporated by reference in its entirety). No other studies followed on these two proteins. Very recently, two yeast (S. cerevisiae) alkaline ceramidases, phytoceramidase (YPC1p) and dihydroceramidase (YDC1p), were cloned and partially characterized by Mao et al., “Cloning of an Alkaline Ceramidase from Saccharomyces Cerevisiae. An Enzyme with Reverse (CoA-independent) Ceramide Synthase Activity,” J. Biol. Chem. 275:6876-6884 (2000), Mao et al., “Cloning and Characterization of a Saccharomyces Cerevisiae Alkaline Ceramidase with Specificity for Dihydroceramide,” J. Biol. Chem. 275:31369-31378 (2000), which are hereby incorporated by reference in their entirety. YPC1p was cloned as a high copy suppressor of the growth inhibition of FB1 as it has fumonisin resistant ceramide synthase activity. The second alkaline ceramidase, YDC1p was identified by sequence homology to YPC1p. A database search reveals that YPC1p and YDC1p are not homologous to any proteins with known functions, but are homologous to putative proteins from Arabidoposis, C. elegans, peptides deduced from EST sequences of human, mouse, pig, zebra fish, and human genomic sequences. A human homologue has been identified and its cDNA has been cloned. Preliminary results show that this human homologue is also an alkaline ceramidase that selectively hydrolyzes phytoCer.

Ceramidase expression according to the present invention relates to the protein level and to expression of the gene encoding the enzyme. Ceramidase activity according to the present invention involves physical and chemical transformation of the enzyme on a particular substrate, as well as the effect of such transformation on target cells or tissue.

Acid ceramidase is expressed in human cumulus cells and follicular fluid, and, in accordance with the present invention, the level of this enzyme can be positively correlated with the quality of human embryos formed in vitro. The present invention discloses an assay kit that can be used for detection of acid ceramidase secreted by IVF-derived embryos into the culture media. According to standard protocols, human embryos derived by IVF are cultured in 50 μl media drops under paraffin oil for 3 days, after which they are then transferred into a fresh 50 μl drop for an additional 2 days. The developmental potential and morphology of the embryos are assessed at these stages (i.e. Day 3 and Day 5) and used to grade the embryos and choose those that are suitable for re-implantation.

In the present invention, 10 μl of media can be collected from the Day 3 and Day 5 drops in which the embryos are cultured and transferred directly into a 96 well ELISA plate for immunodetection of acid ceramidase enzyme secreted by the embryos. Detection is accomplished using a specific monoclonal anti-acid ceramidase antibody detected by a secondary HRP-conjugated antibody and TMB substrate (Sigma). The reaction is stopped by 1N HCl, and product absorbance is assessed at 450 nm using a spectrophotometer. The level of acid ceramidase in the media can serve as a biomarker for the prediction of the developmental potential of the embryos. The present invention includes development of a detection kit to be used by an IVF specialist to classify embryos prior to returning them to the uterus for re-implantation. This assay can be combined with standard methods for morphological grading of IVF derived emrbyos. Briefly, the kit can include an ELISA plate, buffers, and specific antibodies, allowing for immunodetection of acid ceramidase in the embryo exposed media. The kit is a non-invasive method to predict the likelihood that the embryo can be re-implanted and result in a healthy birth.

The method of the present invention preferably involves, without limitation, screening for acid ceramidases.

Recent studies have suggested that ceramide is an important lipid second messenger that induces programmed cell death in oocytes and embryos. Acid ceramidase (AC) is an enzyme that regulates the levels of ceramide, sphingosine, and the pro-survival lipid, sphingosine-1-phosphate (S1P). Mutations in the AC gene (Asah1) result in Farber Lipogranulomatosis, a fatal human disorder. Previous studies showed that AC is highly expressed and active in healthy mouse oocytes and embryos, and that the lack of this enzyme led to embryo death by the 4-cell stage (Eliyahu et al., “Acid Ceramidase Is a Novel Factor Required for Early Embryo Survival,” FASEB J. 21(7):1403-9 (2007), which is hereby incorporated by reference in its entirety). The present invention demonstrates that the levels of AC expression or activity in human oocytes and embryos are associated with good embryo or oocyte quality. Cumulus cells form a multilayered cell mass that support oocyte maturation in vivo. It has been reported that ceramide is translocated from cumulus cells into the adjacent oocyte and induces apoptotic cell death (Perez et al., “A Central Role for Ceramide in the Age-related Acceleration of Apoptosis in the Female Germline,” FASEB J. 19:860-2 (2005), which is hereby incorporated by reference in its entirety). AC expression and activity levels in the cumulus cells could potentially influence the level of ceramide transferred from the cumulus cells into the oocyte. The present invention evaluates the AC expression and activity levels in human post-retrieval cumulus cells as a potential method to assess oocyte quality.

Acid ceramidase (“AC”) is an enzyme that catalyzes the hydrolysis of ceramide to sphingosine and free fatty acid (Bernardo et al., “Purification, Characterization, and Biosynthesis of Human Acid Ceramidase,” J. Biol. Chem. 270(19):11098-102 (1995), which is hereby incorporated by reference in its entirety). Mature AC is a ˜50 kDa protein composed of an α-subunit (˜13 kDa) and a β-subunit (˜40 kDa) (Bernardo et al., “Purification, Characterization, and Biosynthesis of Human Acid Ceramidase,” J. Biol. Chem. 270(19):11098-102 (1995), which is hereby incorporated by reference in its entirety). It is produced through cleavage of the AC precursor protein (Ferlinz et al., “Human Acid Ceramidase: Processing, Glycosylation, and Lysosomal Targeting,” J. Biol. Chem. 276(38):35352-60 (2001), which is hereby incorporated by reference in its entirety), which is the product of the Asah1 gene (NCBI UniGene GeneID No. 427, which is hereby incorporated by reference in its entirety). The present invention demonstrates that AC promotes cell survival.

Acid ceramidases that can be used in the present invention include, without limitation, those set forth in Table 1. In the present invention (including the in vivo methods discussed below), the acid ceramidase can be homologous (i.e., derived from the same species) or heterologous (i.e., derived from a different species) to the one or more embryos or embryonic cells being treated.

TABLE 1
Exemplary Acid Ceramidase Family Members
Homo sapiens

| | |
|---|---|
| UniProt | Q13510, Q9H715, Q96AS2 |
| OMIM | 228000 |
| NCBI Gene | 427 |
| NCBI RefSeq | NP_808592, NP_004306 |
| NCBI RefSeq | NM_177924, NM_004315 |
| NCBI UniGene | 427 |
| NCBI Accession | Q13510, AAC73009 |

Mus musculus

| | |
|---|---|
| UniProt | Q9WV54, Q3U8A7, Q78P93 |
| NCBI Gene | 11886 |
| NCBI RefSeq | NP_062708 |
| NCBI RefSeq | NM_019734 |
| NCBI UniGene | 11886 |
| NCBI Accession | AK151208, AK034204 |

Gallus gallus

| | |
|---|---|
| UniProt | Q5ZK58 |
| NCBI Gene | 422727 |
| NCBI RefSeq | NP_001006453 |
| NCBI RefSeq | NM_001006453 |
| NCBI UniGene | 422727 |
| NCBI Accession | CAG31885, AJ720226 |

Pan troglodytes

| | |
|---|---|
| NCBI Gene | 464022 |
| NCBI RefSeq | XP_519629 |
| NCBI RefSeq | XM_519629 |
| NCBI UniGene | 464022 |

Caenorhabditis elegans

| | |
|---|---|
| UniProt | O45686 |
| IntAct | O45686 |
| NCBI Gene | 173120 |
| NCBI RefSeq | NP_493173 |
| NCBI RefSeq | NM_060772 |
| NCBI UniGene | 173120 |
| NCBI Accession | O45686, CAB05556 |

Danio rerio

| | |
|---|---|
| UniProt | Q5XJR7 |
| NCBI Gene | 450068 |
| NCBI RefSeq | NP_001006088 |
| NCBI RefSeq | NM_001006088 |
| NCBI UniGene | 450068 |
| NCBI Accession | AAH83231, CB360968 |

Rattus norvegicus

| | |
|---|---|
| UniProt | Q6P7S1, Q9EQJ6 |
| NCBI Gene | 84431 |
| NCBI RefSeq | NP_445859 |
| NCBI RefSeq | NM_053407 |
| NCBI UniGene | 84431 |
| NCBI Accession | AAH61540, AF214647 |

Embryo and oocyte cultures according to the present invention can be provided by methods that will be apparent to the skilled artisan.

Embryos and oocytes which can be tested according to the present invention may be obtained from mammalian species. These mammals include, without limitation, embryos from humans, monkeys, mice, rats, guinea pigs, cows, hamsters, sheep, horses, pigs, dogs, and cats.

Screening of the sample, according to the present invention, is performed by screening for ceramidase expression or activity. Ceramidase expression can be screened for by detecting protein levels and levels of expression of nucleic acids encoding ceramidase. Ceramidase activity can be screened for by detecting and measuring the physical and chemical properties related to the enzyme.

The ceramidase according to the present invention includes those identified above. The ceramidase can be in protein form or in the form of a nucleic acid molecule encoding the ceramidase.

Samples of nucleic acids encoding ceramidase (e.g., RNA or corresponding cDNA) from a mammal can be isolated and prepared from tissue or cells using methods known in the art. For example, RNA preparation must produce enzymatically manipulatable mRNA or analyzable RNA. The RNA may be isolated using the guanidinium isothiocyanate-ultracentrifugation method, the guanidinium and phenol-chloroform method, the lithium chloride-SDS urea method, or the poly A+/mRNA from tissue lysates using oligo (dT) cellulose method. It is important that the quality and quantity of RNA yield be accessed prior to quantitative gene analysis. Total isolated RNA can be used to generate first strand copy DNA (cDNA) using any known procedure in the art, for example, using random primers, oligo-dT primers, or random-oligo-dT primers. The cDNA can then be used as a template for a first round amplification reaction or for the quantitative PCR reaction depending on target or sample abundance. The first round PCR amplification is performed with a primer set, including forward and reverse primers, that are specific for the target gene of interest. Following the first round of amplification, a cleaned portion of the reaction product is used for quantitative analysis. Quantitative real-time PCR protocols typically rely on fluorescent detection of product formation following the extension phase of the reaction cycle. Typical fluorescent approaches for quantitative PCR are based on a fluorescent reporter dyes such as SYBR green, FAM, fluorescein, HEX, TET, TAMRA, etc. and quencher dyes such as DABSYL, Black Hole, etc. Systems, such as Molecular Beacons (Integrated DNA Technologies, Coralville, Iowa), Taqman Probes® (Applied Biosystems, Foster City, Calif.), Scorpion® Primers (DxS Ltd., Manchester, UK) are also well known in the art of quantitative gene analysis.

Quantitative gene expression can be expressed as absolute copy number or as relative gene expression. Both methods utilize a standard curve from which to accurately obtain quantitative data. Alternatively, relative gene expression can also be calculated using the Comparative C_{T} Method. The Comparative C_{T} method is similar to the standard curve method, except it uses the arithmetic formula to achieve the same result.

Sample ceramidase protein from a mammal can be isolated and prepared from tissue or cells using standard preparation methods known in the art. For example, tissue and cells can be lysed in buffer containing a detergent, such as sodium dodecyl sulfate (SDS), and a cocktail of protease inhibitors. Protein yield can be determined using the Bradford Assay or any variation of the method known in the art. Assessing the level of expression of a target protein within a sample can be performed by various techniques known in the art, For example, assessing the level of expression can involve analyzing one or more proteins by two-dimensional gel electrophoresis, mass spectroscopy, high performance liquid chromatography (HPLC), fast protein liquid chromatography, multi-dimensional liquid chromatography followed by tandem mass spectrometry, or protein chip expression analysis. Other techniques, using antibodies or other agents which selectively bind to the protein of interest, commonly used for assessing protein expression include Western Blot, immunoprecipitation, enzyme-linked immunosorbent assay (ELISA), radioimmunoassay (RIA), or fluorescent activated cell sorting (FACS). Immunohistochemical and immunofluorescent techniques in which antibody binding to specific protein target is visualized within a whole cell or whole tissue sample is also contemplated.

The sample can be screened for ceramidase activity, protein level, or nucleic acid encoding level by methods that will be apparent to the skilled artisan. Suitable methods include, for example, AC activity assays (Eliyahu et al., “Acid Ceramidase Is a Novel Factor Required for Early Embryo Survival,” FASEB J. 21(7):1403-9 (2007), which is hereby incorporated by reference in its entirety), western blotting to determine the relative amount of AC present in the sample (where a higher amount of AC protein correlates to a higher AC activity level) (Eliyahu et al., “Acid Ceramidase Is a Novel Factor Required for Early Embryo Survival,” FASEB J. 21(7):1403-9 (2007), which is hereby incorporated by reference in its entirety), and RIA (Ferlinz et al., “Human Acid Ceramidase: Processing, Glycosylation, and Lysosomal Targeting,” J. Biol. Chem. 276(38):35352-60 (2001), which is hereby incorporated by reference in its entirety).

The sample according to the present invention may be a liquid used to culture the embryo ex vivo before they are implanted into a recipient female. Alternatively, the sample can be a liquid or cells from a location proximate to an embryo growing in a female subject. For example, such liquids include follicular fluid, amniotic fluid, cumulus cells, and follicule mural granulosa cells. Thus, the present invention affords the ability to assess whether embryo implantation will be successful before or after implantation. In addition, this technique is useful in evaluating an embryo in utero following either in vitro fertilization or natural conception. Alternatively, the present invention can be used to facilitate the identification and selection of healthy embryos for implantation during in vitro fertilization procedures, especially for older human women and for veterinary breeding procedures.

The sample according to the present invention can also be an oocyte or a surrounding liquid. Examples of such liquids include, without limitation, follicular liquid, cumulus cells, or follicule mural granulosa cells. Samples of liquids used in oocyte cryopreservation may also be screened for ceramidase expression and activity. This technique provides a great advantage in the prediction of oocytes likely to provide successful IVF implantation.

The present invention also relates to a kit for predicting the likelihood of embryo or oocyte survival. The kit includes an agent which specifically recognizes ceramidase expression or activity and a label which detects said recognition of ceramidase expression or activity by said agent.

One way of carrying out this aspect of the present invention is with an antibody or binding portion thereof which specifically recognizes ceramidase. Detecting the presence of a complex between an antibody or binding portion thereof and a ceramidase in accordance with the present invention can be carried out by any conventional format for detecting antigen-antibody reactions. Examples include sandwich assays and competitive assays. Formats to detect antigen-antibody reactions are described, e.g., in Klein, Immunology, New York:John Wiley & Sons, pp. 394-407 (1982), which is hereby incorporated by reference. For in vitro detection of embryo/oocyte survival, the formation of a complex between the antibody and ceramidase present in the sample of an embryo or oocyte can be detected by enzyme linked assays, such as ELISA assays. Briefly, the antibody/ceramidase complex is contacted with a second antibody which recognizes a portion of the antibody that is complexed with the ceramidase. Generally, the second antibody is labeled so that its presence (and, thus, the presence of an antibody/ceramidase complex) can be detected. Alternatively, the antibody or binding portion thereof can be bound to a label effective to permit detection of the ceramidase upon binding of the antibody or binding portion thereof to the ceramidase.

Suitable labels include, fluorophores, chromophores, radiolabels, and the like.

According to the present invention, a radiolabeled antibody or binding portion thereof can be used for in vitro diagnostic tests. The specific activity of a tagged antibody or binding portion thereof depends upon the half-life and isotopic purity of the radioactive label and how the label is incorporated into the antibody or its binding portion. Examples of labels useful for detection imaging in accordance with the present invention are radiolabels such as ¹³¹I, ¹¹¹In, ¹²³I, ⁹⁹mTc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The antibody or binding portion thereof can be labeled with such reagents using techniques known in the art. For example, see Wensel and Meares, Radioimmunoimaging and Radioimmunotherapy, New York:Elsevier (1983), which is hereby incorporated by reference, for techniques relating to the radiolabeling of antibodies. See also, Colcher et al., “Use of Monoclonal Antibodies as Radiopharmaceuticals for the Localization of Human Carcinoma Xenografts in Athymic Mice,” Meth. Enzymol. 121:802-816 (1986), which is hereby incorporated by reference.

Instead of detecting the ceramidase protein, nucleic acids encoding this protein can be detected. In this aspect of the present invention, nucleic acid molecules which hybridize under stringent conditions to nucleic acid molecules encoding a ceramidase of the present invention can be used as probes in hybridization assays to detect embryo or oocyte survival in a subject. For example, a sample of embryo/oocyte is contacted with a nucleic acid probe which, under stringent conditions, hybridizes to a nucleic acid molecule encoding a ceramidase according to the present invention. This is carried out under conditions effective to permit formation of a hybridization complex between the probe and the embryo or oocyte specific nucleic acid molecules (i.e., the nucleic acid molecules encoding the ceramidase of the present invention). Embryo/oocyte survival is then determined.

As used herein, the term “probe” refers to an oligonucleotide which forms a duplex structure with a sequence of a target nucleic acid (e.g., a nucleic acid molecule which encodes a ceramidase) due to complementary base pairing. The probe will contain a hybridizing region, which is a region of the oligonucleotide corresponding to a region of the target sequence. A probe oligonucleotide either can consist entirely of the hybridizing region or can contain additional features which allow for the detection or immobilization of the probe but do not alter the hybridization characteristics of the hybridizing region.

The nucleic acid molecules which hybridize under stringent conditions to nucleic acid molecules encoding a ceramidase of the present invention can also be used as primers in a DNA amplification assay to detect survival of the embryo or oocyte in a subject. For example, a sample of embryo or oocyte can be contacted with a nucleic acid primer which, under stringent conditions, hybridizes to a nucleic acid molecule encoding a ceramidase according the present invention or to a complement thereof. The sample of tissue or body fluid from the patient in contact with the nucleic acid primer is then treated under conditions effective to amplify embryo/oocyte specific nucleic acid molecules, and the embryo/oocyte specific nucleic acid molecules, thus amplified, are then detected.

As used herein, the term “primer” refers to an oligonucleotide, whether natural or synthetic, capable of acting as a point of initiation of a DNA synthesis under conditions which produce a primer extension product complementary to a nucleic acid strand. Generally, the DNA synthesis is carried out in the presence of four different nucleoside triphosphates and an agent for polymerization (e,g., DNA polymerase or reverse transcriptase) in an appropriate buffer (e.g., Tris-HCl), and at suitable temperatures (e.g., at an annealing temperature of from about 45 to about 85° C.; at an extending temperature of from about 55 to about 75° C.; and at a melting temperature of about 95° C.). The primer is preferably a single-stranded DNA. The optimal length of the primer depends on the primer's intended use but typically ranges from 15 to 35 nucleotides. Short primer molecules generally require cooler temperatures to form sufficiently stable hybrid complexes with the template. A primer need not complement the exact sequence of the template but must be sufficiently complementary to hybridize with a template. Primers can incorporate additional features which allow for the detection or immobilization of the primer but do not alter the basic property of the primer, that of acting as a point of initiation of DNA synthesis.

The use of PCR to amplify nucleic acid molecules is described in U.S. Pat. No. 4,683,195 to Mullis et al., U.S. Pat. No. 4,683,202 to Mullis, and U.S. Pat. No. 4,965,188 to Mullis et al., which are hereby incorporated by reference.

There are a variety of known methods for determining whether amplification has occurred. For example, a portion of the PCR reaction mixture can be subjected to gel electrophoresis, the resulting gel can be stained with, for example, a ultraviolet absorbing stain, such as with ethidium bromide, and the stained gel can be exposed to ultraviolet light to determine whether a product of the expected size can be observed. Alternatively, labeled PCR primers or labeled deoxyribonucleoside 5′-triphosphates can be used for incorporation of the label into the amplified DNA. The presence of an embryo or oocyte specific nucleic acid amplification product can then be detected by detecting the label.

In a preferred embodiment of the present invention, the cells are eggs and the subject is a female human subjected to chemotherapy after administering the acid ceramidase.

Suitable cell culture media according to the present invention include, without limitation, M2 for oocytes and embryos.

It is expected that embryo cultures with low AC have a higher percentage of apoptotic embryos, and, thus, a poorer predicted outcome for in vitro fertilization. Therefore, the ceramidase activity level of the sample can be correlated to the predicted outcome by comparing that level to a standard level. The standard can be determined using population data from females of various ages.

The present invention may be further illustrated by reference to the following examples.

### EXAMPLES

### Example 1

### Embryo Collection

For a 2-cell embryo collection, superovulated females were caged with males of proven fertility and sacrificed 46 hours after injection of hCG. Embryos were isolated from the oviducal ampullae and cultured at 37° C. in a humidified atmosphere of 5% CO₂ and 95% air.

### Example 2

### Western Blot Analysis

Embryos were subjected to lysis in buffer containing 50 mM Tris-HCL, 150 mM NaCl, 2 mM EDTA, 1% NP-40, 1 mM Vanadate, 5 mM Naf, and 10 μg/ml aprotinine (pH 7.4). Proteins were separated by SDS-PAGE using 10% or 12% pre-cast Nupage Bis/Tris gels under reducing conditions and MES running buffer (Invitrogen), and transferred onto a nitrocellulose membrane (Amersham Biosciences) using a semi-dry transfer apparatus (BioRad) and Nupage-MOPS transfer buffer. For immunoblot analysis, blots were blocked with TBS/Tween containing 5% dry milk, and then were incubated with Goat IgG against acid ceramidase (“AC”) (specific for the β-subunit). Bound antibodies were recognized by secondary antibodies conjugated to horseradish peroxidase. Detection was performed by an enhanced chemiluminescence detection reagent (Amersham Biosciences). Approximate molecular masses were determined by comparison with the migration of pre-stained protein standards (BioRad).

### Example 3

### Acid Ceramidase Activity Assay

Total embryonic cell extracts were incubated for 22 hours at 37° C. with 0.1 ng/ml BODIPY-conjugated C12-ceramide in 0.1M citrate/phosphate buffer (pH 4.5), 150 mM NaCl, 0.05% BSA, and 0.1% Igepal CA-630. After the reactions were complete, 5 μl of the assay mixtures were removed and added into 95 μl of ethanol, mixed, and then centrifuged for 5 minutes at 10,000×g. The supernatants were then transferred to a Waters glass sampling vial, and 5 μl (2.5% of the original reaction mixture) were auto-sampled by a WIPS 712 (Waters) autosampler onto a high performance liquid chromatograph equipped with a reverse-phase column (BetaBasic-18, 4.6×30 mm, Keystone Scientific Inc., Bellefonte, Pa.), and eluted isocratically with methanol/water (95:5 v/v) at a flow rate of 1 ml/min. Fluorescence was quantified using a Waters 474 fluorescence detector set to excitation and emission wavelengths of 505 nm and 540 nm, respectively. The undigested substrate (i.e., BODIPY-conjugated C12-ceramide) and product (i.e. fatty acid) peaks were identified by comparing their retention times with standards, and the amount of product was calculated using a regression equation that was established from a standard curve using BODIPY-conjugated C12 fatty acid.

### Example 4

### Data Presentation and Statistical Analysis

All experiments were independently replicated at least three times. The combined data from the replicate experiments were subjected to a t-test analysis, and results were considered statistically significant at P<0.005. Graphs represent the mean±s.e.m. of combined data from the replicate experiments.

### Example 5

### Acid Ceramidase Expression in Unfertilized Mouse Oocytes

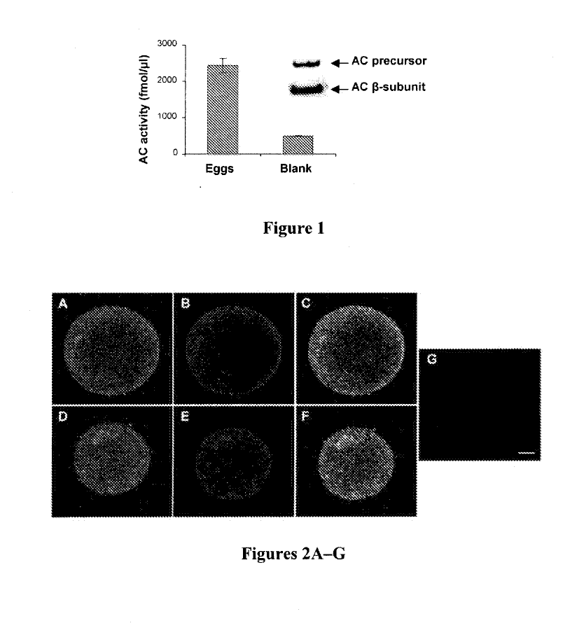

Cell extracts were prepared from 400 pooled, unfertilized MII eggs (collected 16 hours after hCG injection), and analyzed by western blot to identify the AC protein. As can be seen in FIG. 1, the AC precursor protein (55 kDa) and β-subunit (40 kDa) are expressed in the egg before fertilization. The presence of the processed β-subunit indicates that some of the AC was likely to be active. Cell extracts were therefore prepared from an additional 65 pooled, unfertilized eggs, and subjected to AC activity assays. As shown in FIG. 1, these analyses revealed a high enzymatic activity (t-test, p<0.005), confirming the western blot results.

To obtain information about the subcellular location of AC in eggs, immunohistochemistry was performed using anti-AC specific antibodies combined with anti-LAMP1 staining for late endosome/lysosome detection. The fluorescence distribution of the AC and LAMP-1 signals was visualized at the equator and cortex of the egg, and photographed with a Ziess confocal laser-scanning microscope, as shown in FIGS. 2A-G. These studies reveal that AC is localized mainly at the egg cortex, as shown in FIGS. 2A and 2D, and co-localizes with LAMP-1 in the late endosomes/lysosomes, as shown in FIGS. 2B and 2D-F.

### Example 6

### Acid Ceramidase Expression and Localization in Human Oocytes and Early Embryos

Oocytes and embryos from women scheduled for in vitro fertilization with intracyctoplasmic sperm injection were collected ˜32 hours (oocytes) or 3-5 days (embryos) after injection of luteinizing hormone.

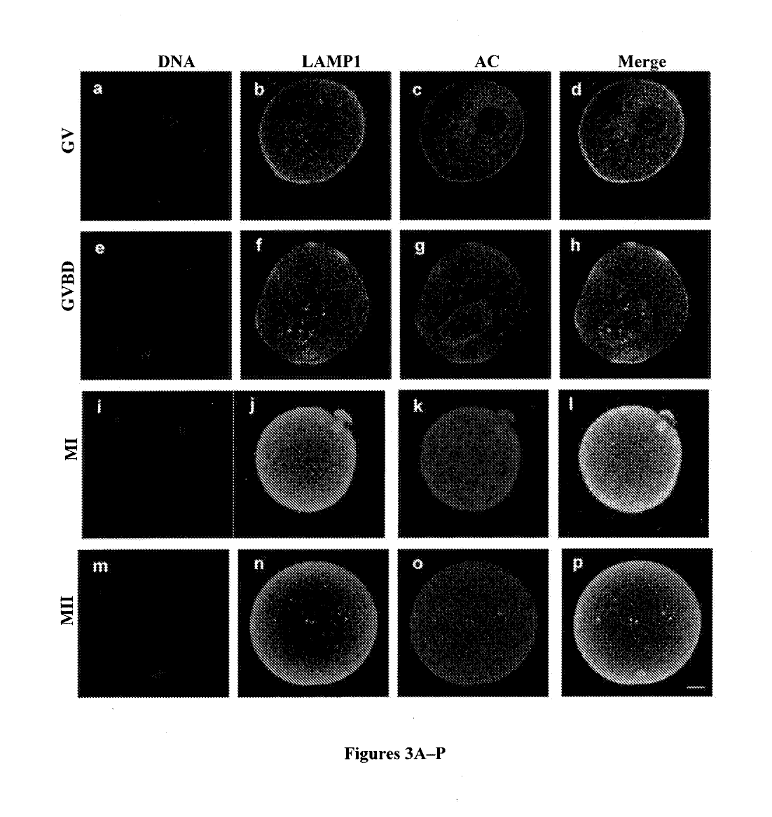

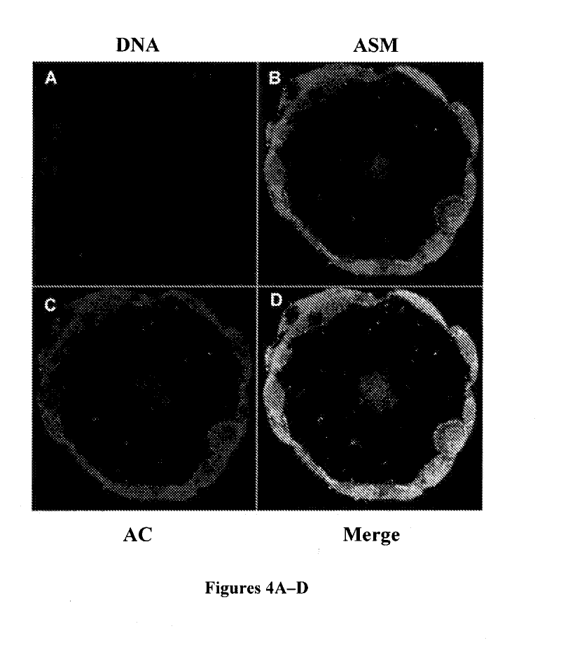

Co-immunohistochemistry assays were performed to detect the localization and possible interaction between AC and lysosome associated membrane protein (“LAMP”) (a lysosomal enzyme marker) during human oocyte maturation. Oocytes were triple labeled for AC protein, cellular DNA, and LAMP, and examined for co-localization by immunofluorescence confocal microscopy. As shown in FIGS. 3A-H, AC is localized mainly in the cortex and membrane, and co-localizes with LAMP, during both the germinal vesicle stage (FIGS. 3A-D) and the germinal vesicle break down stage (FIGS. 3E-H). In addition, AC is co-localized with the GV membrane break down, as shown in FIGS. 3E-H. As shown in FIGS. 3I-L, AC protein is co-localized with LAMP and with DNA during the MI stage. During the MII stage, AC is homogenously distributed throughout the cytosol with a marked localization at the membrane and cortex, and co-localization in the spindle, as shown in FIGS. 3M-P). These data clearly show changes in the developmental pattern of AC expression during human egg maturation. This is the first known study that demonstrates that AC is expressed in human oocytes.

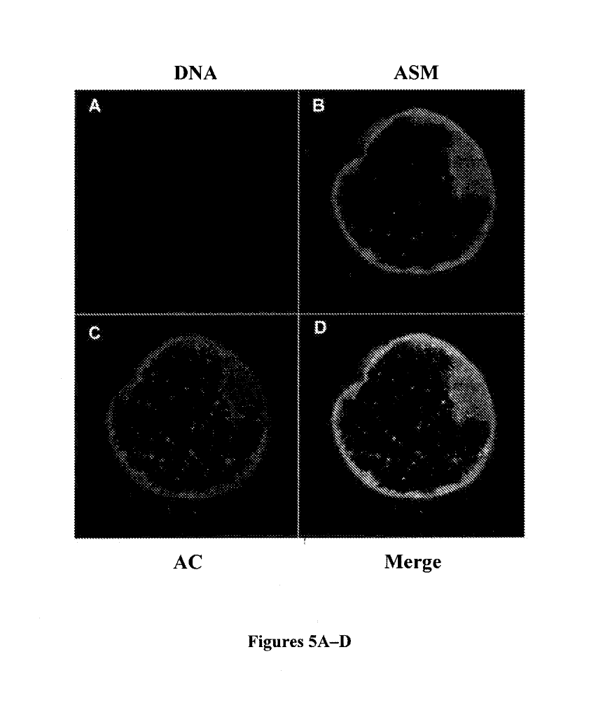

Co-immunohistochemistry assays were also performed to detect the localization and possible interaction between AC and acid sphingomyelinase (“ASM”), a related enzyme that hydrolyzes sphingomyelin into ceramide, during early embryo development. Embryos were triple labeled for AC protein, cellular DNA, and ASM, and examined for co-localization by immunofluorescence confocal microscopy. As shown in FIGS. 4A-D and FIGS. 5A-D, AC is localized in the embryonic fluid and co-localizes with ASM mainly in the inner and outer cell mass. Moreover, high-grade embryos (FIGS. 5A-D) demonstrate higher expression of AC in the embryonic fluid than low-grade embryos (FIGS. 4A-D). Thus, high-grade embryos would be expected to have lower ceramide levels and higher S1P levels than low-grade embryos, and, therefore, a higher survival rate (due to a lower incidence of apoptosis).

### Example 7

### Acid Ceramidase Expression and Activity in Human Follicular Fluid

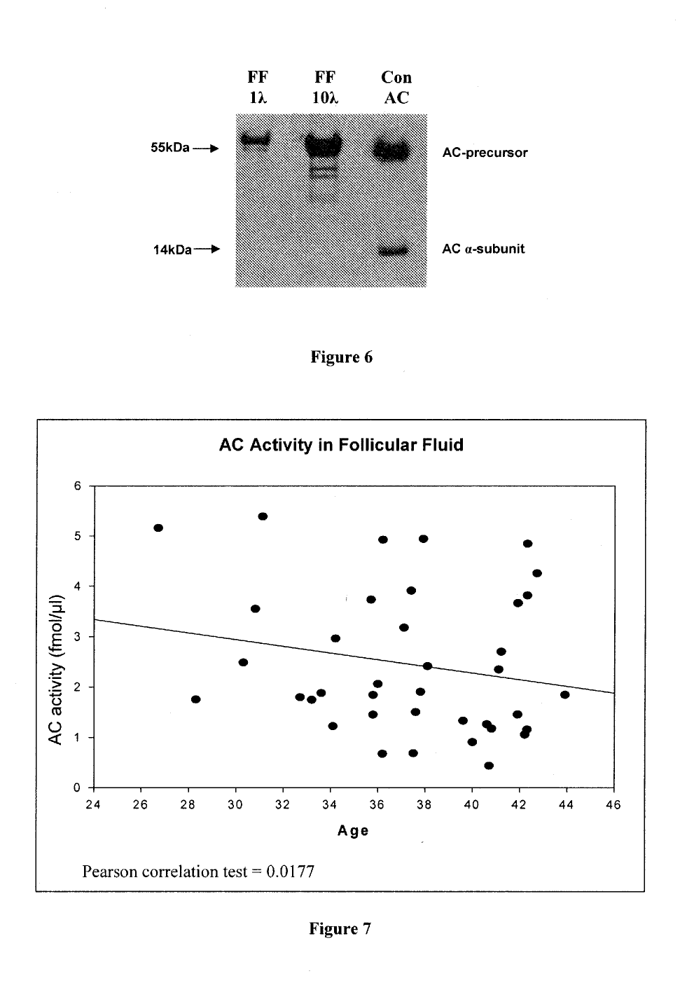

Human follicular fluid samples from oocytes assigned for in vitro intracyctoplasmic sperm injection were collected during oocyte retrieval. Western blot analysis was used to evaluate the total amount of AC in the follicular fluid. Proteins were separated by SDS-PAGE. A monoclonal mouse anti-human AC IgM was used to detect the AC precursor protein (55 kDa). As shown in FIG. 6, the AC precursor protein is highly expressed in human follicular fluid.

An in vitro activity assay was used to evaluate the activity of AC in human follicular fluid. Follicular fluid samples were incubated under acidic conditions for 22 hours at 37° C. with BODIPY conjugated C12-ceramide, and then analyzed by HPLC. The results of this activity assay were correlated with patient age using the Pearson correlation test. As shown in FIG. 7, there is a trend towards a decrease in AC activity with increasing age. This suggests that the measurement of AC in follicular fluid can be used as a marker for reproductive aging.

### Example 8

### Measurement of AC in Embryo Culture

Human embryos were created by in vitro fertilization and grown for 5 days in 50 microliters of culture media. At the end of the culture period, the media was removed, centrifuged, and acid ceramidase (AC) levels were determined using HPLC-based activity assays.

Ten media samples (from 10 human embryos) have been assessed for AC activity. These embryos were all derived from same woman (and sperm donor). This woman was referred to the Reproductive Medicine Associates (RMA) for a family history of spontaneous abortion and a specific chromosomal defect (fragile X syndrome).

Of the 10 embryos studied, 4 were not developed. The average AC activity in the media of these 4 embryos was 223 units/microliter. Two of 10 embryos were judged to be “high quality” by microscopy, and their average activity was 369 units/microliter. These data confirm the hypothesis according to which higher AC activity is compatible with higher quality embryos (due to lower levels of the lipid, ceramide). Of the 4 remaining embryos, 2 were at the “blastocyst” stage, and 2 were at the later morula stage. The average activity in the blastocyst embryos was 281 units/microliter, and the average activity in the morula embryos was 406 units/microliters.

### Example 9

### Preimplantation Embryos

Ten preimplantantion embryos were obtained from the same egg and sperm donors following IVF. They were cultured for 4 days in 50 microliters of standard IVF media and assessed according to their developmental stage and morphology. Four embryos were undeveloped (arrested), 3 had reached the blastocyst stage, and 3 had reached the morula stage. Single cells were removed and used for gender determination and to assess Fragile X Syndrome (due to a prior family history). Based on these findings, two of the embryos were transferred into the recipient.

The media was removed from each embryo and used to determine acid ceramidase activity. The experiment was performed “blindly”, without knowledge of the embryo quality or outcome. There were three important outcomes of this experiment: 1) acid ceramidase activity could be detected in media from such early stage embryos (i.e., the present methods were sensitive enough); 2) later stage embryos (morula) had higher levels of activity than earlier stage embryos (blastocyst); and 3) developed embryos had higher activity than arrested embryos. The results are shown in Table 2.

TABLE 2
Comparative Activities of Morula, Blastocysts,
Developed and Arrested Embryos

| | | Genetic Result | |
|---|---|---|---|
| AC | Developmental | For Fragile X & | |
| Activity | Stage | Gender | Outcome |
| 311.10 | Blastocyst | Affected Female | |
| 251.99 | Blastocyst | Failed Amplification | |
| 381.66 | Blastocyst | Normal Male | Transferred |
| 255.95 | Morula | Affected Male | |
| 357.55 | Morula | Normal Female | Transferred |
| 557.96 | Morula | Affected Female | |
| 261.43 | Arrested | Normal Female | |
| 10 | Arrested | Affected Male | |
| 232.23 | Arrested | Affected Male | |
| 390.75 | Arrested | Normal Male | |

Morula versus Blastocyst: 391 versus 314 fmol/microliter
Developed versus Arrested: 357 versus 294 fmol/microliter

These results show that as embryos develop in vitro, they express and release more acid ceramidase into the culture media (morula vs. blastocyst). They also show that healthy (developed) embryos of the same stage release more acid ceramidase than unhealthy embryos (arrested).

### Example 10

### AC Activity Measurement in Cumulus Cells

Human cumulus cells were obtained from 33 IVF patients who gave consent to use discarded materials for research. The average patient age was 35.7±4.3 years old and ranged from 28 to 42.8. Oocytes were removed from cumulus cells and cultured for insemination. The average number of oocytes retrieved was 15.2±7.5 and ranged from 3 to 32. Cumulus cells from each individual patient were collected and centrifuged to form a pellet. Excess media were removed from the sample prior to freezing. Protein extract was prepared using thawed cumulus cells and CelLyticM total cell lysis buffer (Sigma), according the manufacturer instructions. Wilcoxon signed-rank test was performed using MicroSoft Analyze-It. Variables included AC activity levels in cumulus cells, patient age, the number of retrieved oocytes, oocyte maturity and peak serum estradiol levels.

AC activity was detected in 24 samples. The mean activity level was 273.2±168.5 pmol/μl and ranged from 34.5 to 593.5 pmol/μl. In 13 cases, the maturity of the retrieved oocytes was available on the day of retrieval. The numbers of Metaphase II oocytes and AC levels in the post-retrieval cumulus cells were significantly associated (p=0.005). Patient age was significantly related to the number of retrieved oocytes (p<0.001), and the number of oocytes retrieved per patient was associated with AC activity levels in post-retrieval cumulus cells (p<0.001). The association between age and AC activity in cumulus cells per oocyte was also significant (p<0.001). Finally, AC activity levels were associated significantly with peak serum estradiol values (p<0.001) and the success of embryo implantation by in vitro fertilization. These results are summarized in Table 3.

TABLE 3
Correlation of AC Activity in Post-Retrieval Cumulus Cells

| | | Significance (Wilcoxon |
|---|---|---|
| | | non-parametric paired |
| Variable | Correlation | groups) |
| AC Activity & | Older the Woman, | p < 0.005 |
| Maternal Age | Lower the AC | |
| AC Activity & | More Oocytes Retrieved, | p < 0.001 |
| # Oocytes Retrieved | Higher the AC | |
| AC Activity & Peak | Higher Hormone Levels, | p < 0.001 |
| Estrogen Levels | Higher the AC | |
| Pregnancy & AC | Successful Pregnancy | p < 0.001 |
| | After Implantation, | |
| | Higher the AC | |

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed:

1. A method of predicting the likelihood of embryo or oocyte survival, said method comprising:
providing a sample of an embryo, an oocyte, or surrounding liquid or cells;
screening the sample for ceramidase expression or activity; and
correlating the ceramidase expression or activity obtained through said screening to a prediction of the likelihood of embryo or oocyte survival.

2. The method of claim 1, wherein the ceramidase is an acid ceramidase.

3. The method of claim 1, wherein the embryo or the oocyte, or the surrounding liquid is from a mammal.

4. The method of claim 3, wherein the mammal is selected from the group consisting of monkey, mice, rats, guinea pigs, hamsters, horses, cows, sheep, pigs, dogs, and cats.

5. The method of claim 1, wherein said screening is conducted for ceramidase activity level.

6. The method of claim 1, wherein said screening is conducted for ceramidase protein levels.

7. The method of claim 1, wherein said screening is conducted for expression of a nucleic acid encoding ceramidase.

8. The method of claim 1, wherein the sample is a liquid used to culture the embryo or an oocyte ex vivo.

9. The method of claim 1, wherein the sample is a liquid from a female subject.

10. The method of claim 1, wherein the sample is an embryo that was produced by in vitro fertilization.

11. The method of claim 1, wherein the sample is an embryo that was produced by natural conception.

12. The method of claim 1, wherein the sample is an oocyte.

13. A kit for predicting the likelihood of embryo or oocyte survival, said kit comprising:
an agent which specifically recognizes ceramidase expression or activity and
a label which detects said recognition of ceramidase expression or activity by said agent.

14. The kit of claim 13, wherein the ceramidase is an acid ceramidase.

15. The kit of claim 13, wherein the embryo or the oocyte, or the surrounding liquid is from a mammal.

16. The kit of claim 15, wherein the mammal is selected from the group consisting of monkey, mice, rats, guinea pigs, hamsters, horses, cows, sheep, pigs, dogs, and cats.

17. The kit of claim 13, wherein said agent detects ceramidase activity level.

18. The kit of claim 13, wherein said agent detects ceramidase protein levels.

19. The kit of claim 13, wherein said agent detects expression of a nucleic acid encoding ceramidase.

21. The kit of claim 13, wherein said agent which recognizes ceramidase expression or activity is an antibody.
